(19) United States
(12) Patent Application Publication
(10) Pub. No.: US 2015/0112237 A1
(43) Pub. Date: Apr. 23, 2015

(54) DEVICE FOR REHABILITATING BRAIN MECHANISM OF VISUAL PERCEPTION USING COMPLEMENTARY SENSUAL STIMULATIONS

(71) Applicant: Yissum Research Development Company of The Hebrew University of Jerusalem LTD., Jerusalem (IL)

(72) Inventor: Amir Amedi, Jerusalem (IL)

(73) Assignee: Yissum Research Development Company of The Hebrew Jerusalem LTD., Jerusalem (IL)

(21) Appl. No.: 14/396,548

(22) PCT Filed: Apr. 23, 2013
(86) PCT No.: PCT/IL2013/050349

§ 371 Date: Oct. 23, 2014

Related U.S. Application Data
(60) Provisional application No. 61/637,039, filed on Apr. 23, 2012.

(51) Int. Cl. A61F 9/08; A61F 11/00; A61H 23/00; A61N 1/36

(52) U.S. Cl. CPC A61F 9/08; A61N 1/3606; A61F 11/00; A61H 23/00
USPC 601/47; 607/3

(57) ABSTRACT

A system for both rehabilitating vision related neural tissue and augmenting vision perception with higher resolution and color information is provided herein. Both rehabilitation and augmentation are achieved by providing direct neural stimulation and at the same time an auditory/tactile signal that serves as an explanatory signal aiding the visual perception by easily perceived sensations. The system includes a capturing device configured to capture a video sequence of images of a scene; a processing unit configured to: (i) convert visual data derived from each captured image into at least one of: auditory representation and tactile representation; and (ii) convert the visual data of a respective captured image into an neural stimulation representation; a neural stimulator configured to generate neural stimulation onto a vision related neural tissue; and an output unit configured to generate an auditory stimulation and tactile output based on the auditory representation and tactile representation, respectively.

### BACKGROUND

1. Technical Field

The present invention relates to visual rehabilitation, and more specifically, to visual rehabilitating (of visually impaired) and augmentation (e.g. of high-resolution color information) of the visual perception in the brain by applying sensual stimulations of different types.

2. Discussion of the Related Art

The use of neural stimulation as a means for rehabilitating neural and brain mechanisms is well known in the art. For example, clinical researches have shown recovery and rehabilitation of injured or dysfunctional visual systems. Some researchers have shown that a deliberate stimulation of neural tissues in which the stimulation represents visual data of an actual scene may help to develop the neural connections and revive the visual perception capabilities of the brain and the vision related neural system at least to some (quite limited) extent.

Sensory substitution devices are non-invasive human-machine interfaces which transform visual information into auditory or tactile inputs, and enable the blind to “see” using their other senses, offering, in principle, high-resolution visual information. One challenge is how sensory substitution devices can be used to provide high visual resolution, or high visual acuity. Another challenge is how congenitally blind individuals, who have never experienced sight, can learn to perceive high-resolution ‘vision’.

Several attempts were directed at restoring vision to the blind. One approach is by applying an electro-neural stimulation to vision nerves via electrodes, wherein the stimulation is based on a conversion of captured visual data into neural stimulation. Specifically, a device preferably worn on the head of a visually impaired person captures images of the scene which in turn are converted to electro-neural stimulation that is applied, via a set of electrodes, to the relevant portion of the neural tissue.

While some improvement in patients using the aforementioned device has been demonstrated, the limited number of electrodes yields unsatisfactory results. It should be noted however, that while increasing the number of electrodes is possible (from less than a hundred to over a thousand, for example) there is an inherent limitation of resolution in using a set of electrodes, due to the impracticality of coupling a very large number of vision nerves (of any type) with the electrodes. Another critical limitation is that the brain do not always (or usually) knows how to interpret the information arriving from the electrical stimulation as the brain was dramatically changed after years (or lifelong) of blindness. Despite the aforementioned attempts, the currently available sensory substitution devices provide poor resolution while other solutions lack the ability to provide the patient with a real perception of vision.

### BRIEF SUMMARY

According to some embodiments of the present invention, a system for both rehabilitating vision related neural tissue and augmenting visual perception with high resolution and color information is provided. The system may include a capturing device configured to capture a video sequence of images of a scene; a processing unit configured to: (i) convert visual data derived from each captured image into at least one of: auditory representation and tactile representation, based on a first mapping; and (ii) convert the visual data of a respective captured image into a neural stimulation representation, based on a second mapping; a neural stimulator configured to generate neural stimulation onto a vision related neural tissue, wherein the neural stimulation is based on the neural stimulation representation; and an output unit configured to generate at least one of: an auditory stimulation and tactile output, based on the auditory representation and tactile representation respectively, wherein the neural stimulation, the auditory stimulation, and the tactile stimulation are carried out within a specified timeframe.

According to some embodiments of the present invention, a method of achieving both rehabilitation of vision related neural tissue and augmentation of visual perception with high resolution and color information is provided herein. The method may include the following stages: converting visual data derived from each captured image into an auditory or tactile representation, based on a first mapping; converting the visual data of a respective captured image into a neural stimulation representation, based on a second mapping; generating a neural stimulation, wherein the neural stimulation is based on the neural stimulation representation; generating an auditory or tactile stimulation, based on the auditory representation and tactile representation respectively; and applying within a specified timeframe: (1) the neural stimulation to a vision related neural tissue of the person and (2) the auditory stimulation and/or tactile stimulation to ears and portions of skin of the person, respectively, wherein the applying is usable for the several therapeutic purposes. For example, some therapeutic purposes may include: (i) rehabilitating the vision related neural tissue; (ii) developing vision-auditory neural pathways; (iii) developing vision-tactile neural pathways; (iv) providing alternative visual orientation; (v) providing an explanatory information for the neural stimulation; and (vi) providing increased resolution, color information and context to the neural stimulation which provides the visual qualia, being the feeling of vision. The latter outcome is particularly advantageous for late blinds people (which forms the great majority of blind worldwide).

Advantageously, embodiments of the present invention overcome the drawbacks of the existing approaches for visual rehabilitation. All existing approaches lack the ability to recover color vision which is critical not only for enhancement of the experience but is further crucial for enhancing the ability to recognize objects in natural settings and separate figures from background which is key computation needed for vision. Embodiments of the present invention thus create a hybrid system in which a first component stimulates or recovers the peripheral visual system in a bottom up manner to recover gray scale low-resolution qualia of vision (it is not possible so far to create systematic stimulation to achieve color vision) and a second component adds high-resolution perception and color information top-down using sounds and touch information.

These, additional, and/or other aspects and/or advantages of the embodiments of the present invention are set forth in the detailed description which follows; possibly inferable from the detailed description; and/or learnable by practice of the embodiments of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1 is a high level schematic block diagram illustrating an environment of a system according to some embodiments of the invention;

FIG. 2 is a high level schematic block diagram illustrating the system according to some embodiments of the invention;

FIGS. 3A-3D are signal diagrams illustrating an aspect according to some embodiments of the invention;

FIG. 4 is a high level flowchart illustrating a method according to some embodiments of the invention;

FIG. 5 show images illustrating one aspect according to some embodiments of the invention;

FIG. 6 is a table illustrating another aspect according to some embodiments of the invention; and

FIG. 7 show a comparison between images illustrating an aspect according to some embodiments of the invention.

The drawings together with the following detailed description make apparent to those skilled in the art how the invention may be embodied in practice.

### DETAILED DESCRIPTION

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

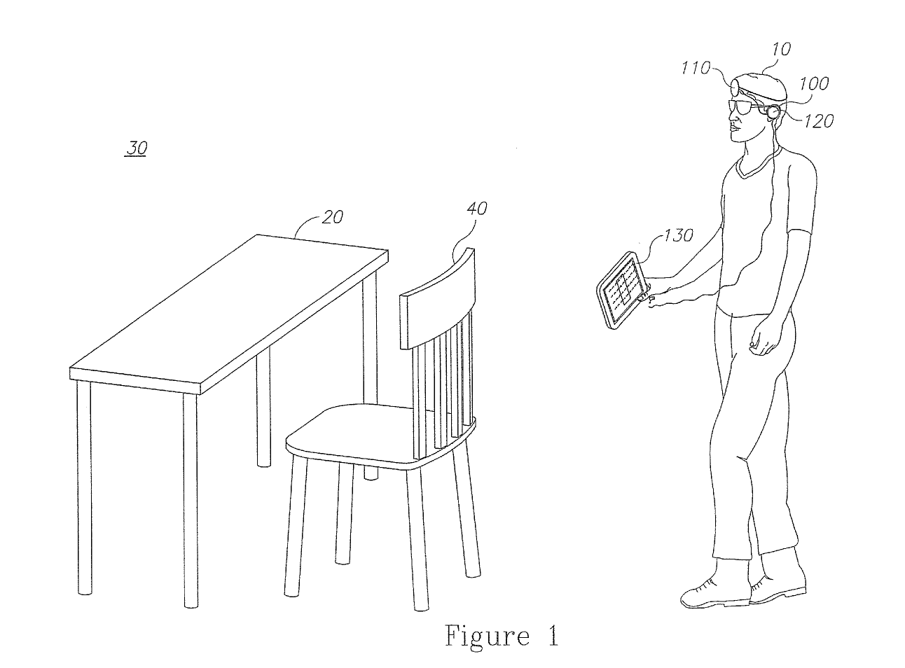

FIG. 1 is a high level schematic block diagram illustrating an environment of a system according to some embodiments of the invention. A scene 30 may include objects such as a table 20 and a chair 40. A blind or visually impaired person 10 wears system 100 which includes an image capturing device 110 such as a miniature webcam connected by wire to system 100. Additionally, earphones 120 are connected to system 100 and so is a tactile output unit 130 such as a matrix of pins. Further a set of electrodes (not shown) are coupled to a specified portion of the neural tissue of the visual system of person 10.

Embodiments of the present invention present a combination of neural stimulation together with either auditory or tactile stimulation (or both) for the purposes of arousing visual rehabilitation and reconstruction of original neural pathways. Additionally, the combined stimulation may further provide the blind patient with explanatory information for the neural stimulation. Another outcome is providing increased resolution, color and context to the neural stimulation which provides the visual qualia, being the feeling of vision.

The combining is correlated in a specified faun such that the two or three types of stimulation are useable to develop visual pathways in the central neural system. System 100 does so by capturing real-time images by a image capturing device 110 into a series of instrumental sound sweeps or tactile stimuli that covers the entire image in two seconds or less, together with matrix of electrophysiological stimulation electrodes which are capable of stimulating visual areas and so reflecting an image directly to the nerve system by using appropriately calibrated electrical stimulation, which fits the visual data.

The inventors have discovered that continuously applying electrical stimulation to the visual nerve system such that the electrical stimulation is correlated with a synchronous sensory stimulation wherein each one of the stimulations is a transformation of the visual data, will significantly increase the speed of rehabilitation in the visual system. The rate of rehabilitation of the visual nerve system using system 100 is significantly higher than the rate of rehabilitation of the visual nerve system with each one of the stimulations taken alone. This end is achieved since the auditory and/or tactile signal provide an explanatory signal to the direct neural stimulation. Put differently, the auditory and/or the tactile stimulations serve as a so-called “sensual interpreter” to the neural stimulation applied directly to the neural tissues. Advantageously, this combined stimulation takes advantage of the regenerative nature of the neural pathways in the brain.

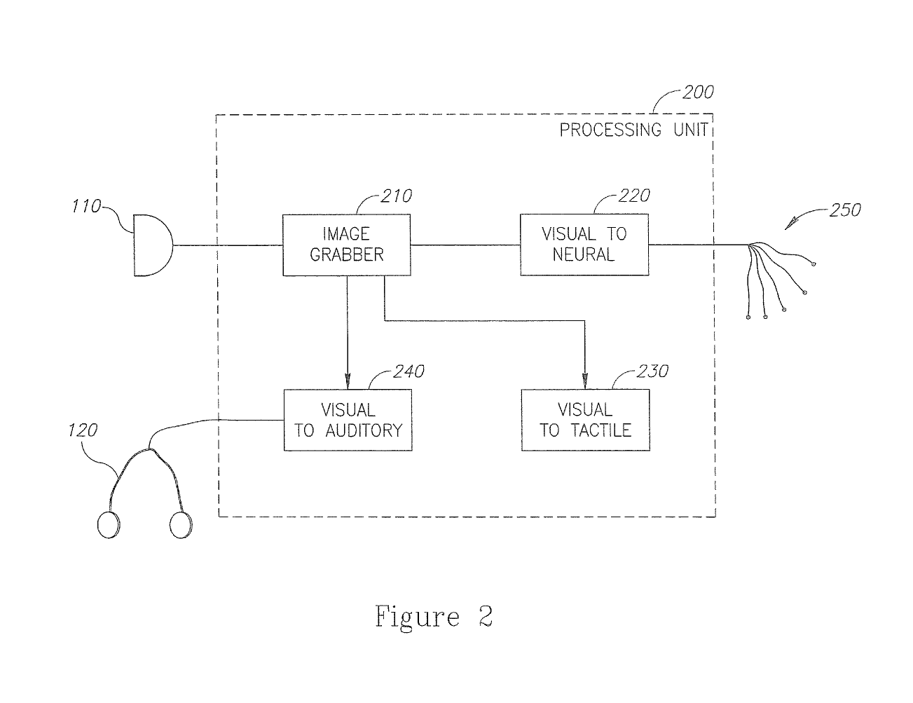

FIG. 2 is a high level schematic block diagram illustrating the system according to some embodiments of the invention. The system includes a capturing device 110 configured to capture a video sequence of images of a scene. The system further includes a processing unit 200 configured to: (i) grab images from the capturing device 100 using an image grabber 210; (ii) convert visual data derived from each captured image into at least one of: an auditory representation using visual to auditory mapping 240 and a tactile representation using visual to tactile mapping 230; and (iii) convert the visual data of a respective captured image into a neural stimulation representation, using a visual to neural mapping 220. The system further includes a neural stimulator 250, such as a set of electrodes, configured to generate neural stimulation onto a vision related neural tissue. Specifically, the neural stimulation is based on the neural stimulation representation

Finally, the system further includes output unit such as earphones 120 configured to generate an auditory stimulation and a tactile output unit 130 such as, but not necessarily, a matrix of pins configured to generate a tactile stimulation. Both auditory stimulation and tactile stimulations are based on the auditory representation and tactile representation respectively. Thus, person 10 is presented with several representations of the same visual image. These stimulations can be used for several purposes. Some of the demonstrated applications are: (i) rehabilitating the vision related neural tissue; (ii) developing vision-auditory neural pathways; (iii) developing vision-tactile neural pathways; (iv) providing alternative visual orientation; (v) providing an explanatory information for the neural stimulation; and (vi) providing increased resolution and context to the neural stimulation which provides the visual qualia, being the feeling of vision. This latter outcome is particularly advantageous for late blinds people.

The inventors have discovered that by using complementary sensual stimulations, the advantages in terms of perception achieved by auditory or tactile stimulation are used to enhance the visual perception via the direct neural stimulation to the neural tissue of the visual system.

Consistent with one embodiment of the invention, the neural stimulator comprises a plurality of electrophysiological electrodes configured to convey electrophysiological signals. An exemplary neural stimulator may be in the form of a plurality of electrophysiological electrodes set in an N×M matrix layout. It is understood that other neural stimulators may be used in order to carry out the present invention.

In order to increase the affectivity of the cross-sensual stimulation, the neural stimulation, the auditory stimulation, and the tactile stimulation are carried out either simultaneously or within a specified timeframe of 1-2 seconds. Additionally, both the auditory stimulation and the tactile stimulation may be carried out in a form of a swipe or a scan of scene 30 so that person 10 receives these complementary stimulations over a period of time such as 0.5 to 2 seconds. It is understood that both delay time and scan time may be adjusted to fit the needs of the patients and further according to the treatments results.

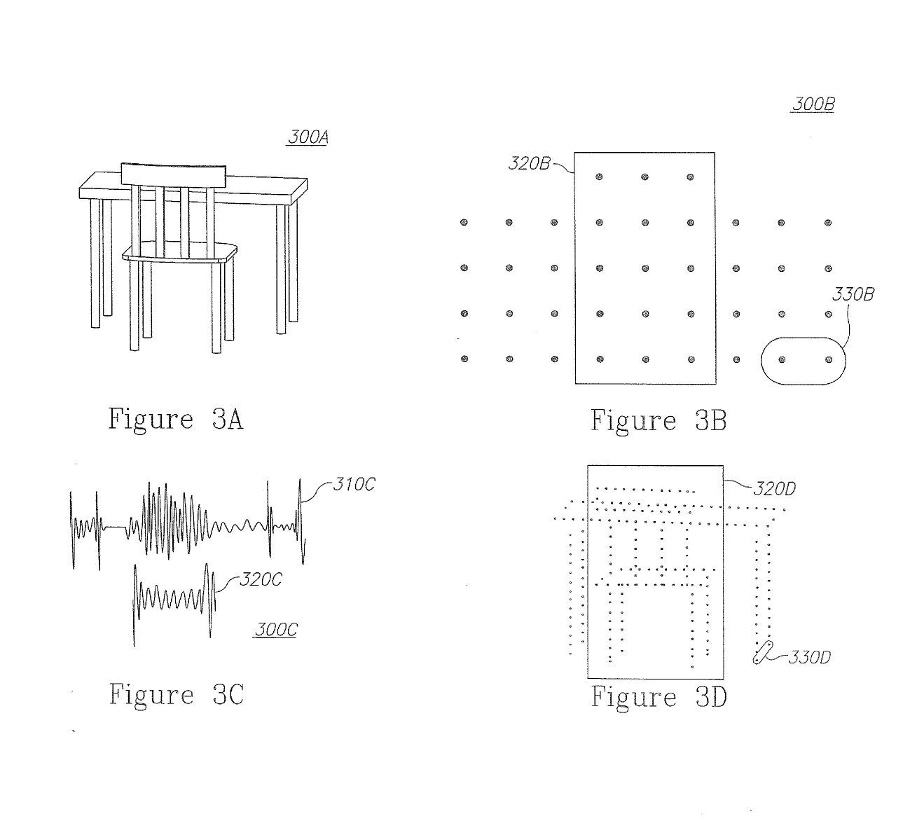

FIGS. 3A-3D are signal diagrams illustrating an aspect according to some embodiments of the invention. Specifically, FIG. 3A shows a visual image of scene 30 as captured by image grabber 210. FIG. 3B shows a neural stimulation scheme 300B represented by electrical stimulation via electrodes in an exemplary 6×10 matrix layout, wherein 320B relates to the chair and 330B relates to the lower tips of the right legs of the table. FIG. 3C shows an exemplary auditory stimulation 300C over time. As shown, auditory waveforms representative of the visual data are presented along a timescale. In the exemplary representation, 310B represents the table while 320B represents the chair. Lastly, FIG. 3D shows a tactile stimulation scheme 300D implemented as a pin matrix and the captured table and chair, wherein 330D represents the two lower tips of the right legs of the table.

Consistent with one embodiment of the invention, the first mapping (associated with auditory/tactile stimulation) is selected such the auditory representation and/or the tactile representation exhibit a resolution within a specified range containing the resolution of the neural stimulation representation. Thus, the resolution of the neural stimulation may be set to either a higher or lower level than the resolution level of the auditory/tactile stimulation, in accordance with specific needs of the patients. In some cases, as may be shown in FIGS. 3B and 3D, the resolution of the tactile stimulation is selected to be significantly higher (e.g., at least 10 times higher) than the resolution of the neural stimulation representation.

Similarly the auditory signal can be up to two orders of magnitude higher than the neuronal stimulation if needed. This too can be adjusted according to the performance and needs of the patient, first start with lower resolution and as performance improved increase resolution. This feature is advantageous in providing higher spatial resolution. For example, both 330B and 330D represent the two lower tips of the right legs of the table. However, in 330D the location and orientation of the legs is much more apparent than in 330B. Carrying out embodiments of the present invention will help to enhance the visual perception caused by the neural stimulation, with better spatial resolution and shape perception.

Consistent with one embodiment of the invention, the first mapping and the second mapping are adjustable in quality and quantity and may be selected such that any of the following applications: (i) rehabilitating of the vision related neural tissue, (ii) developing of the vision-auditory neural pathways, and (iii) developing of the vision-tactile neural pathways are maximized. The optimization may be carried out prior to treatment based on the patient's data or may be adjusted in response to the treatment results. Alternatively, the optimization may be carried in real-time during treatment and based on actual feedback from the patient.

According to some embodiments of the present invention, the system may further include a capturing device configured to capture said visual data from a scene. The capturing device can capture either still image or video sequences. Alternatively, the visual data may be derived from a restored retina of a patient. It is understood however, that the capturing device is not necessarily part of the systems and the visual data may be provided from an external source.

As can be seen in FIG. 3C the auditory representation of the visual data converts at least one of: shape, position, and color of objects in the scene, into at least one of: type of waveform, level of pitch, and type of musical instrument. Similarly, further data indicative of a distance can be also represented by appropriate auditory representation. The distance information may be obtained using a distance sensor such as IR sensor supplying information indicative of a distance of an object at the light of sight. The distance information may further enhance the distance vision perception in the rehabilitation process and in the vision augmentation

Thus, table 310B and chair 320B may be represented by different musical instruments, different pitch or other means for auditorily distinguishing that in turn may help person 10 in distance perception of the neural stimulation so as to distinguish as shown in FIG. 3B between the chair 320B and the table 310B. The auditory stimulation may also be advantageous in enhancing distance perception and separation of figure (persons or objects) from its noisy background one of the most difficult tasks in vision.

By providing auditory signal indicative of color in the scene, the patient is provided with more visual information than can be provided by currently available sensory substitution devices which provide black & white only representation. Thus, the auditory-color representation not only enhances the “vision” experience but also provides valuable information of the scene, such as colors of a traffic light.

Consistent with one embodiment of the invention, first mapping is selected such that at least one of: shape and texture of objects contained within the visual data are converted into the tactile representation, while at least one of: shape, position and color of objects contained within the visual data are converted into the auditory representation.

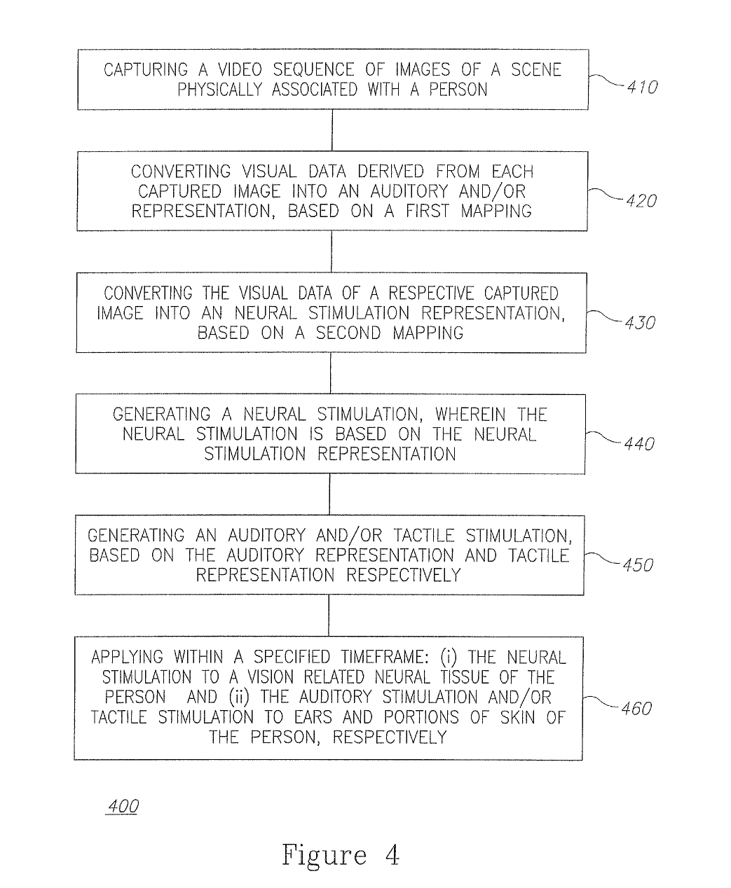

FIG. 4 is a high level flowchart illustrating a method 400 according to some embodiments of the invention. It is understood that stages of method 400 may be carried out independently of the aforementioned architecture of system 100. Method 400 begins with the stage of capturing a video sequence of images of a scene physically associated with a person 410. It then goes on to the stage of converting visual data derived from each captured image into auditory or tactile representation, based on a first mapping 420 and to the stage of converting the visual data of a respective captured image into a neural stimulation representation, based on a second mapping 430. Then, method 400 proceeds to the stages of generating a neural stimulation, wherein the neural stimulation is based on the neural stimulation representation 440 and to the stage of generating an auditory or tactile stimulation, based on the auditory representation and tactile representation respectively 450. Finally, method 400 goes on to the stage of applying within a specified timeframe: (i) the neural stimulation to a vision related neural tissue of the person and (ii) the auditory stimulation and/or tactile stimulation to ears and portions of skin of the person, respectively.

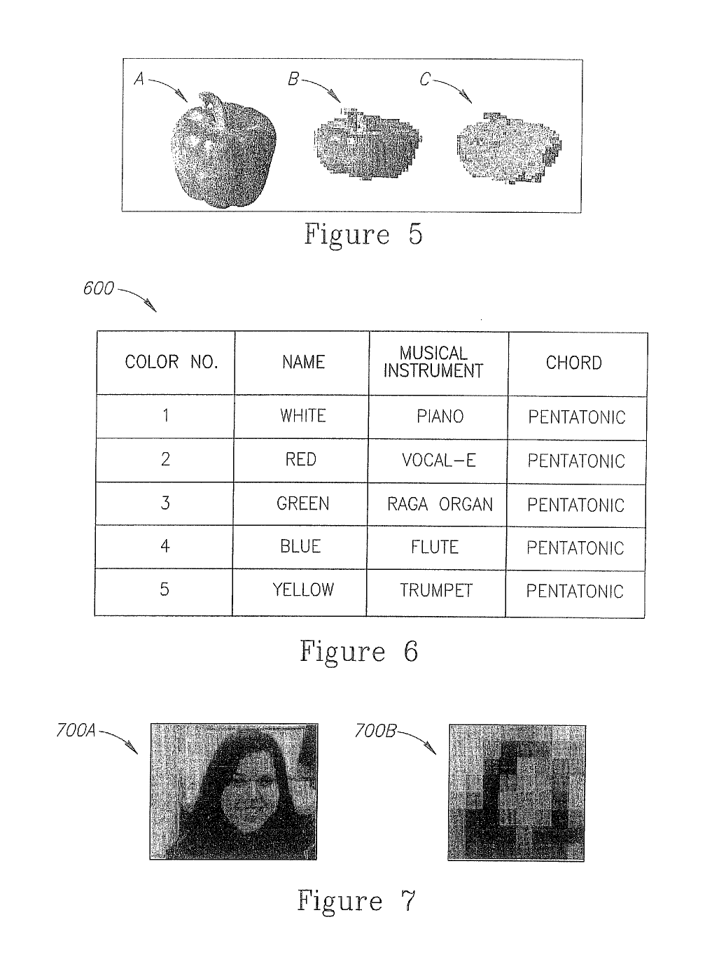

FIG. 5 shows images A, B, and C illustrating an example for how the representation and augmentation of color data is carried out, according to some embodiments of the invention. Specifically, in representing the visual data captured, some focus may be put on the color data contained therein. The introduction of color data may both augment the vision perception of the blind and facilitate the rehabilitation process. In order to represent the color data, the following exemplary process may be carried out: a captured image A shows an object in full color. Image A is converted into a resolution-degraded image B which shows an image with a similar color gamut as image A but with a substantially reduced resolution. The reduced resolution may be adjusted to the physical limitations of either the audio-tactile output unit or the neural stimulator described above. Then, image B is converted into image C showing the object both with the reduced resolution and further using only a subset of colors selected from the original color gamut. Again, the selection of this subset is carried out in order to meet the physical limitations of both the audio-tactile output unit and the neural stimulator. Each of the color of the subset of colors of image C are then mapped into an auditory representation that may me based on exemplary table shown in FIG. 6, in which the green color is represented by a Raga organ and the blue color is represented by a flute. It is understood that it is possible to code in this method tens of various shades of colors by adding more instruments for instance, but here we demonstrate only the four main colors plus black and white.

The inventor has discovered that by carefully selecting the subset of colors and their respective auditory representation, better results in terms of color perception of the blind persons may be achieved. For example, assigning specified musical instrument chords for specified colors may improve the ability to distinguish between colors and the use of chords rather than pure tones may improve the ease of learning, the pleasantness of the sounds (which might have been a hard limitation on sensory substitution devices according to the prior art) and the overall training process.

The inventors have discovered that by carrying out embodiments of the present invention, blind patients can retrieve visual information in much higher resolution than was previously demonstrated by any of the sight rehabilitation alternatives either sensory substitution devices (SSDs) or prostheses available today, even following a relatively limited training of 30-100 hours. By way of illustration, FIG. 7 shows two images comparing the resolution between the system according to the prior 700B (such as the chronic vision implant) and an image captured by the system according to the present invention 700A. The significant difference in the captured image is indicative of the significant difference in the effect in both rehabilitation and augmentation capabilities between the system of the prior art and the system of the present invention.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

While the invention has been described with respect to a limited number of embodiments, these should not be construed as limitations on the scope of the invention, but rather as exemplifications of some of the preferred embodiments. Other possible variations, modifications, and applications are also within the scope of the invention.

1. A system comprising:
a processing unit configured to: (i) convert visual data into at least one of: auditory representation and tactile representation, based on a first mapping; and (ii) convert the visual data of a respective captured image into a neural stimulation representation, based on a second mapping;
a neural stimulator configured to generate neural stimulation onto a vision related neural tissue, wherein the neural stimulation is based on said neural stimulation representation; and
an output unit configured to generate at least one of: an auditory stimulation and tactile output, based on said auditory representation and tactile representation respectively,
wherein said neural stimulation, said auditory stimulation, and said tactile stimulation are carried out within a specified timeframe and are usable at least for using the at least one of said auditory stimulation and said tactile stimulation as explanatory signals for said neural stimulation.

2. The system according to claim 1, wherein said first mapping is based at least partially on assigning a unique auditory or tactile representation for each one of a color of a subset of colors representative of all colors contained in said visual data of the captured image.

3. The system according to claim 1, wherein said first mapping is selected such at least one of said auditory representation and tactile representation exhibit a resolution within a specified range containing a level of a perceived resolution of the neural stimulation, wherein at least one of said auditory representation and tactile representation further provide an additional sensual property not provided by the neural stimulation.

4. The system according to any of claims 1 to 4, further comprising a capturing device configured to capture said visual data from a scene.

5. The system according to any of claims 1 to 4, wherein the visual data is derived from a restored retina of a patient.

6. The system according to any of claims 1 to 4, wherein said neural stimulator comprises a plurality of electrophysiological electrodes configured to convey electrophysiological signals.

7. The system according to claim 4, wherein the plurality of electrophysiological electrodes are set in a form of one or more arrays.

8. The system according to any of claims 1 to 4, wherein the first mapping and the second mapping are selected such that at least one of: the rehabilitating of the vision related neural tissue, the developing of the vision-auditory neural pathways, and the developing of the vision-tactile neural pathways is maximized.

9. The system according to any of claims 1 to 4, wherein the first mapping is selected such that the auditory representation of the visual data converts at least one of: texture, shape, position, distance of object at a line of sight, and color of objects in the scene, into at least one of: type of waveform, level of pitch, and type of musical instrument.

10. The system according to any of claims 1 to 4, wherein the first mapping is selected such that the tactile representation of the visual data converts at least one of: texture, shape, position, and color of objects in the scene, into at least one of: type of waveform, level of pitch, and type of tactile actuator.

11. A method comprising:
converting visual data of a scene into auditory or tactile representation, based on a first mapping;
converting the visual data into a neural stimulation representation, based on a second mapping;
generating a neural stimulation, wherein the neural stimulation is based on said neural stimulation representation;
generating an auditory or tactile stimulation, based on said auditory representation and tactile representation respectively; and
applying within a specified timeframe: (i) said neural stimulation to a vision related neural tissue of the person and (ii) said auditory stimulation and/or tactile stimulation to ears and portions of a skin of a human, respectively,
wherein the applying is usable for at least one of: rehabilitating said vision related neural tissue, developing vision-auditory neural pathways, developing vision-tactile neural pathways, and providing alternative visual orientation.

12. The method according to claim 11, wherein said first mapping is based at least partially on assigning a unique auditory or tactile representation for each one of a color of a subset of colors representative of all colors contained in the visual data.

13. The method according to claim 11 or 12, further comprising capturing a video sequence of images of a scene physically associated with a person, wherein said visual data is achieved by the capturing.

14. The method according to claim 11, wherein said visual data is derived from a restored retina.

15. The method according to claim 11, wherein the first mapping is selected such that at least one of the auditory representation and tactile representation exhibit a resolution within a specified range containing a level of a perceived resolution of the neural stimulation, wherein at least one of said auditory representation and tactile representation further provide an additional sensual property not provided by the neural stimulation.

16. The method according to claim 11, wherein the first mapping and the second mapping are selected such that at least one of: the rehabilitating of the vision related neural tissue, the developing of the vision-auditory neural pathways, and the developing of the vision-tactile neural pathways is maximized.

17. The method according to claim 11, wherein the first mapping is selected such that the auditory representation of the visual data converts at least one of: shape, position, distance of object at a line of sight, and color of objects in the scene, into at least one of: type of waveform, level of pitch, and type of musical instrument.

18. The method according to claim 11, wherein the first mapping is selected such that the tactile representation of the visual data converts at least one of: texture, shape, position, and color of objects in the scene, into at least one of: type of waveform, level of pitch, and type of tactile actuator.
